(19) United States
(12) Patent Application Publication
(10) Pub. No.: US 2025/0198981 A1
(43) Pub. Date: Jun. 19, 2025

(54) DETECTION DEVICE

(71) Applicant: Japan Display Inc., Tokyo (JP)

(72) Inventors: Kaoru ITO, Tokyo (JP); Akihiko FUJISAWA, Tokyo (JP); Daichi ABE, Tokyo (JP)

(21) Appl. No.: 18/983,553

(22) Filed: Dec. 17, 2024

(30) Foreign Application Priority Data
Dec. 18, 2023 (JP) 2023-213143

(51) Int. Cl. G01N 33/483

(52) U.S. Cl. CPC G01N 33/4833

(57) ABSTRACT

According to an aspect, a detection device includes: a sensor panel having a detection area in which optical sensors are two-dimensionally arranged; a light source configured to emit light; a member on which an object to be detected is to be placed such that the object to be detected is interposed between the detection area and the light source; and a control circuit configured to perform processing based on outputs of the optical sensors. The member has a light-transmitting area in which the object to be detected is to be placed and a light-blocking area provided around the light-transmitting area. The detection area is located so as to overlap both the light-transmitting area and the light-blocking area. The control circuit is configured to obtain a difference between the outputs of the optical sensors overlapping the light-transmitting area and the outputs of the optical sensors overlapping the light-blocking area.

### CROSS-REFERENCE TO RELATED APPLICATION

This application claims the benefit of priority from Japanese Patent Application No. 2023-213143 filed on Dec. 18, 2023, the entire contents of which are incorporated herein by reference.

### BACKGROUND

### 1. Technical Field

What is disclosed herein relates to a detection device.

### 2. Description of the Related Art

Detection devices are known that enable detection of states of culture environments for culturing biological tissues or microorganisms using an optical sensor (for example, Japanese Patent Application Laid-open Publication No. 2005-087005 (JP-A-2005-087005)).

Sensing of the culture environments by a detection device such as the one in JP-A-2005-087005 is based on the fact that the brightness of light detected by the optical sensor tends to decrease as the culture of an object to be cultured progresses. Therefore, if errors occur in the output of the optical sensor due to external noise or the like, the accuracy of sensing decreases. Detection devices that can reduce such a decrease in accuracy of sensing are required.

For the foregoing reasons, there is a need for a detection device that can restrain the accuracy of sensing from decreasing.

### SUMMARY

According to an aspect, a detection device includes: a sensor panel that has a detection area in which a plurality of optical sensors are two-dimensionally arranged; a light source configured to emit light; a member on which an object to be detected is to be placed such that the object to be detected is interposed between the detection area and the light source; and a control circuit configured to perform processing based on outputs of the optical sensors. The member has a light-transmitting area in which the object to be detected is to be placed and a light-blocking area provided around the light-transmitting area. The detection area is located so as to overlap both the light-transmitting area and the light-blocking area. The control circuit is configured to obtain a difference between the outputs of the optical sensors overlapping the light-transmitting area and the outputs of the optical sensors overlapping the light-blocking area.

### BRIEF DESCRIPTION OF THE DRAWINGS

FIG. 1 is a diagram illustrating a main configuration of a detection device;

FIG. 2 is a diagram illustrating a configuration example of a detection area and a wiring area;

FIG. 3 is a circuit diagram illustrating a circuit configuration of an optical sensor;

FIG. 4 is a schematic diagram schematically illustrating a configuration example of a detection system;

FIG. 5 is a schematic diagram illustrating a relation between one detection device and an external configuration;

FIG. 6 is a schematic view illustrating a positional relation between the main configuration of the detection device and an object to be detected;

FIG. 7 is a schematic view illustrating an object irradiated by light from a sensor panel in plan view;

FIG. 8 is a schematic graph illustrating an exemplary relation of the output of the detection area with a lapse of time associated with a growth of a colony in a culture medium formed on the object to be detected;

FIG. 9 is a schematic view illustrating a mechanism for distinguishing a light-transmitting area from a light-blocking area;

FIG. 10 is a flowchart of processing performed in the detection device;

FIG. 11 is a schematic view illustrating an example of the number of blocks in the detection area and the number of inputs to each multiplexer;

FIG. 12 is a diagram illustrating an exemplary individual detection process based on combinations of the blocks with the inputs to each multiplexer;

FIG. 13 is a diagram illustrating a case where noise NS occurs in the execution of the process illustrated in FIG. 12;

FIG. 14 is a flowchart of processing performed in the detection device according to a first modification of the embodiment;

FIG. 15 is a flowchart of processing performed in the detection device according to a second modification of the embodiment;

FIG. 16 is a schematic view illustrating a configuration example of a light source; and

FIG. 17 is a circuit diagram illustrating a circuit configuration of the optical sensor having a partially different configuration from that of FIG. 3.

### DETAILED DESCRIPTION

The following describes an embodiment of the present disclosure with reference to the drawings. What is disclosed herein is merely an example, and the present disclosure naturally encompasses appropriate modifications easily conceivable by those skilled in the art while maintaining the gist of the present invention. To further clarify the description, the drawings may schematically illustrate, for example, widths, thicknesses, and shapes of various parts as compared with actual aspects thereof. However, they are merely examples, and interpretation of the present disclosure is not limited thereto. The same element as that illustrated in a drawing that has already been discussed is denoted by the same reference numeral through the description and the drawings, and detailed description thereof may not be repeated where appropriate.

In this disclosure, when an element is described as being “on” another element, the element can be directly on the other element, or there can be one or more elements between the element and the other element.

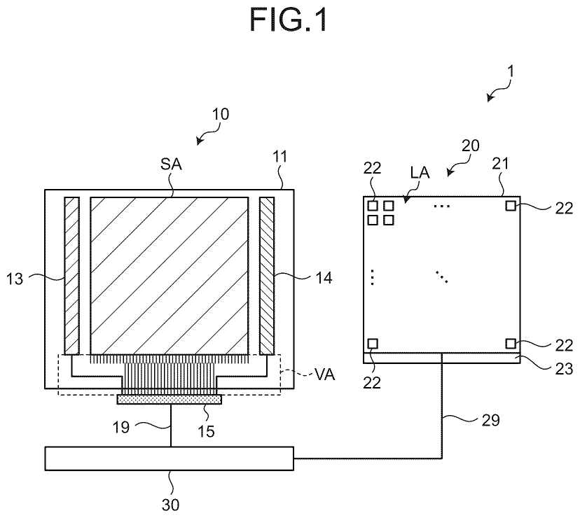

FIG. 1 is a diagram illustrating a main configuration of a detection device 1. The detection device 1 includes a sensor panel 10, a light source panel 20, and a control circuit 30. The sensor panel 10 and the light source panel 20 of the detection device 1 are coupled to the control circuit 30.

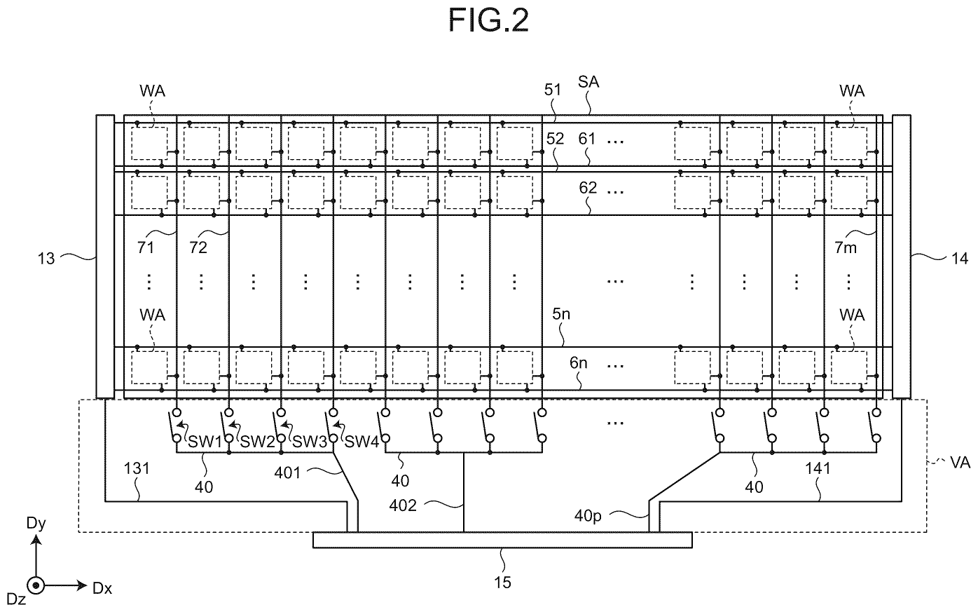

The sensor panel 10 is provided with a detection area SA (refer to FIG. 2) on a substrate 11. A reset circuit 13, a scan circuit 14, and a wiring area VA are mounted on the substrate 11. Components on the detection area SA, the reset circuit 13, and the scan circuit 14 are coupled to a detection circuit 15 via the wiring area VA.

The light source panel 20 has a light-emitting area LA that emits light to the detection area SA. The light source panel 20 is provided with light sources 22 on a substrate 21. The light sources 22 each include a light-emitting element such as a light-emitting diode (LED) and are provided in the light-emitting area LA. In the example illustrated in FIG. 1, a plurality of the light sources 22 are arranged in a matrix having a row-column configuration on the substrate 21.

The light source panel 20 is provided with a light source drive circuit 23. Under the control of the control circuit 30, the light source drive circuit 23 controls turning on and off each of the light sources 22 and the luminance thereof when being turned on. The light sources 22 may be provided so as to be individually controllable in light emission or may be provided so as to emit light all together.

The control circuit 30 performs various types of control related to the operation of the detection device 1. Specifically, the control circuit 30 is a circuit, such as a field-programmable gate array (FPGA) or an application-specific integrated circuit (ASIC) that can implement a plurality of functions. The control circuit 30 is coupled to the detection circuit 15 via a wiring part 19 and obtains the output from the detection circuit 15. The control circuit 30 is coupled to the light source drive circuit 23 via a wiring part 29 and performs processing related to the lighting of the light sources 22, such as determination of lighting patterns of the light sources 22.

The control circuit 30 performs calculation of a difference value and a determination process based on the difference value. The calculation of the difference value and the determination process will be described later.

Although not illustrated in the drawings, the detection device 1 includes, for example, an analog-to-digital conversion circuit and a digital-to-analog conversion circuit. The analog-to-digital conversion circuit allows the output from an optical sensor WA (refer to FIG. 2) transmitted via the detection circuit 15 to be handled in arithmetic processing by the control circuit 30. The digital-to-analog conversion circuit allows digital signals generated by the arithmetic processing of the control circuit 30 to be used to control operations of the sensor panel 10 and the light source panel 20. These circuits may be included, for example, in part or in whole in the control circuit 30, may be functions performed by circuits mounted on a flexible printed circuits (FPCs) provided as the wiring part 19 and the wiring part 29, or may be implemented in other ways in the detection device 1.

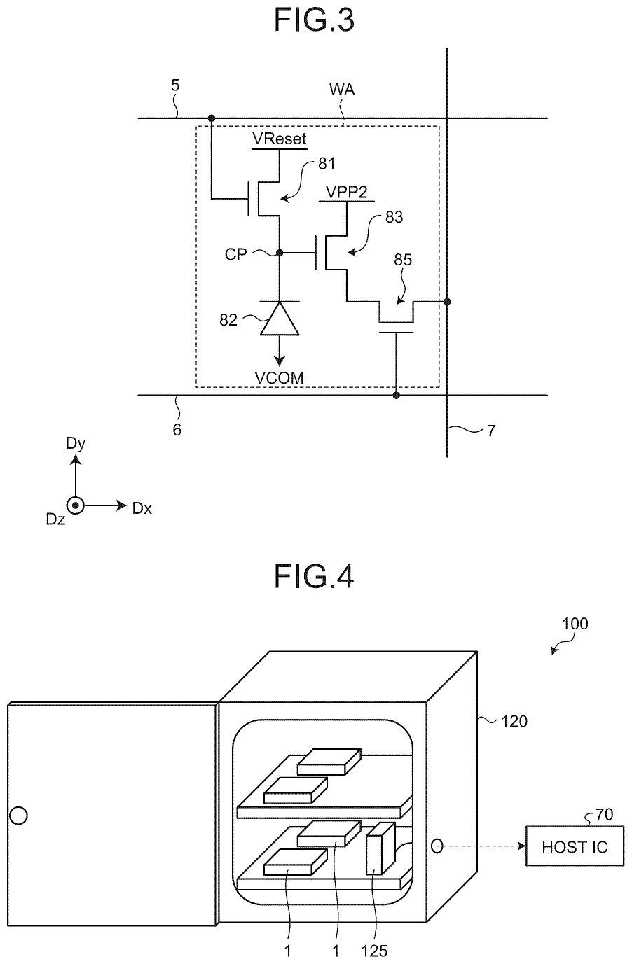

FIG. 2 is a diagram illustrating a configuration example of the detection area SA and the wiring area VA. A plurality of the optical sensors WA (FIG. 3) are provided in the detection area SA. In the embodiment, as illustrated in FIG. 2, the optical sensors WA are arranged in a matrix having a row-column configuration along a first direction Dx and a second direction Dy. The first direction Dx is orthogonal to the second direction Dy. In the following description, the term “third direction Dz” refers to a direction orthogonal to the first direction Dx and the second direction Dy.

The reset circuit 13 is coupled to reset signal transmission lines 51, 52, . . . , 5n. Hereafter, the term “reset signal transmission line 5” refers to any one of the reset signal transmission lines 51, 52, . . . , 5n. The reset signal transmission line 5 is wiring along the first direction Dx. In the example illustrated in FIG. 2, n reset signal transmission lines 5 are arranged in the second direction Dy. n is a natural number equal to or larger than 2. The n reset signal transmission lines 5 are each coupled, at one end in the first direction Dx, to the reset circuit 13.

The scan circuit 14 is coupled to scan lines 61, 62, . . . , 6n. Hereafter, the term “scan line 6” refers to any one of the scan lines 61, 62, . . . , 6n. The scan line 6 is wiring along the first direction Dx. In the example illustrated in FIG. 2, n scan lines 6 are arranged in the second direction Dy. The n scan lines 6 are each coupled, at the other end in the first direction Dx, to the scan circuit 14.

As illustrated in FIG. 2, the reset signal transmission lines 5 and the scan lines 6 are alternately arranged in the second direction Dy in the detection area SA. The reset circuit 13 and the scan circuit 14 illustrated in FIGS. 1 and 2 are arranged at locations facing each other with the detection area SA interposed therebetween, but the layout of the reset circuit 13 and the scan circuit 14 is not limited to this layout and can be changed as appropriate.

Signal lines 71, 72, . . . , 7m are also provided in the detection area SA. Hereafter, the term “signal line 7” refers to any one of the signal lines 71, 72, . . . , 7m. The signal line 7 is wiring along the second direction Dy.

In the example illustrated in FIG. 2, m signal lines 7 are arranged in the first direction Dx. m is a natural number equal to or larger than 2. The m signal lines 7 are each coupled, at one end in the second direction Dy, to one of a plurality of switches (for example, a switch SW1, a switch SW2, a switch SW3, or a switch SW4) included in a multiplexer 40.

The multiplexer 40 is provided in the wiring area VA. The multiplexer 40 includes a plurality of switches. In the example illustrated in FIG. 2, the switches SW1, SW2, SW3, and SW4 are illustrated as the switches. The switches included in one multiplexer 40 are turned on (conducting state) at different times from one another. During a period when one of the switches included in one multiplexer 40 is on (conducting state), the other switches are off (non-conducting state). The number of the multiplexers 40 corresponds to the number (m) of the signal lines 7. When the number of the switches is p, m/p is sufficient as the number of the multiplexers 40. When more than one multiplexer 40 are provided, each of the multiplexers 40 is coupled to the detection circuit 15 via an individual one of wiring lines 401, 402, . . . , 40p.

The coupling between the signal lines 7 and the detection circuit 15 via the multiplexer 40 is merely exemplary and is not limited to this example. The signal lines 7 may be individually directly coupled to the detection circuit 15 in the wiring area VA. In the wiring area VA, the reset circuit 13 is coupled to the detection circuit 15 via wiring 131. In the wiring area VA, the scan circuit 14 is coupled to the detection circuit 15 via wiring 141.

In the detection of light by a PD 82 (refer to FIG. 3) provided in the optical sensor WA, the detection circuit 15 controls the operation timing of the reset circuit 13 and the scan circuit 14. The detection circuit 15 receives the output from the optical sensor WA. The detection circuit 15 converts signals received from the optical sensors WA into data that can be interpreted by the control circuit 30 and outputs the data to the control circuit 30. The detection circuit 15 of the embodiment is a micro-controller unit (MCU).

FIG. 3 is a circuit diagram illustrating a circuit configuration of the optical sensor WA. The first direction Dx and the second direction Dy in FIG. 3 merely correspond to the directions of the reset signal transmission lines 5, the scan lines 6, and the signal lines 7, and do not exactly indicate the relative positional relation of the circuit configuration in the optical sensor WA.

As illustrated in FIG. 3, a switching element 81, the PD 82, a transistor element 83, and a switching element 85 are provided in the optical sensor WA. The PD 82 is a photodiode (PD). The switching elements 81 and 85 and the transistor element are metal-oxide semiconductor field-effect transistors (MOSFETs).

The gate of the switching element 81 is coupled to the reset signal transmission line 5. One of the source and the drain of the switching element 81 is provided with a reset potential VReset. The other of the source and the drain of the switching element 81 is coupled to the cathode of the PD 82 and the gate of transistor element 83. Hereafter, the term “coupling part CP” refers to a point where the other of the source and the drain of the switching element 81 is coupled to the cathode of the PD 82 and the gate of transistor element 83. A reference potential VCOM is provided from the anode side of the PD 82. The potential difference between the reset potential VReset and the reference potential VCOM is set in advance, but the reset potential VReset and the reference potential VCOM may be variable. The reset potential VReset is higher than the reference potential VCOM.

The drain of the transistor element 83 serving as a source follower is provided with a source-of-output potential VPP2. The source of the transistor element 83 is coupled to one of the source and the drain of the switching element 85. The other of the source and the drain of the switching element 85 is coupled to the signal line 7. The gate of the switching element 85 is coupled to the scan line 6.

The reset potential VReset, the reference potential VCOM, and the source-of-output potential VPP2 are supplied by the detection circuit 15 to the optical sensor WA based on, for example, electric power supplied via a power supply circuit (not illustrated) coupled to the detection circuit 15, but are not limited to being supplied in this way, and may be supplied in a different way as appropriate.

The source-of-output potential VPP2 is set in advance. The potential on the source side of the transistor element 83 is a potential lower than the output potential of the PD 82 by a voltage (Vth) between the gate and the source of the transistor element 83. In this case, the potential on the source side of the transistor element 83 corresponds to the reset potential VReset and the reference potential VCOM. The potential of the output of the PD 82 corresponds to the photovoltaic power generated by the PD 82 and corresponding to the light detected by the PD 82 during an exposure period.

When the gate of the switching element 85 is turned on by a signal provided from the scan circuit 14 via the scan line 6, the source and the drain of the switching element 85 are brought into a conducting state therebetween. This operation transmits, to the signal line 7 via the switching element 85, a signal (potential) transmitted via the transistor element 83 to the switching element 85. Thus, the output from the optical sensor WA is generated. Hereafter, the term “scan signal” refers to the signal (potential) provided from the scan circuit 14 via the scan line 6. The scan circuit 14 is a circuit that outputs the scan signal.

The output of one PD 82 provided in one optical sensor WA corresponds to the intensity of the light detected by the PD 82 during the exposure period set in advance. The output of the PD 82 is reset in response to a signal provided by the reset circuit 13 via the reset signal transmission line 5. When the signal turns on the gate of the switching element 81, the source and the drain of the switching element 81 are brought into a conducting state therebetween. This operation resets the potential of the coupling part CP to the reset potential VReset.

FIG. 4 is a schematic diagram schematically illustrating a configuration example of a detection system 100 including the detection device 1. As illustrated in FIG. 4, the detection system 100 includes a plurality of the detection devices 1, a host integrated circuit (IC) 70, and a coupling circuit 125. The detection devices 1 are electrically coupled to the common host IC 70 via the coupling circuit 125.

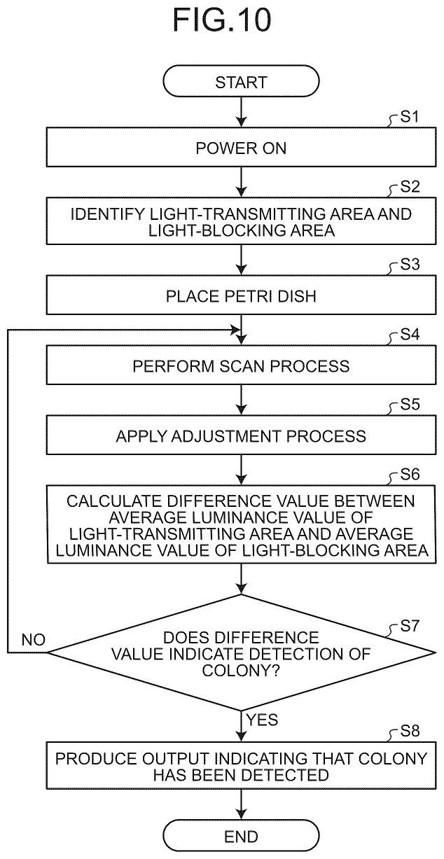

An incubator 120 illustrated in FIG. 4 is maintained such that an environment (temperature, humidity, and the like) therein is suitable for culturing an object to be detected SUB while a door is closed. The detection devices 1 are placed in the incubator 120 and each perform a scan process (refer to FIG. 10 and other figures) to be described later.

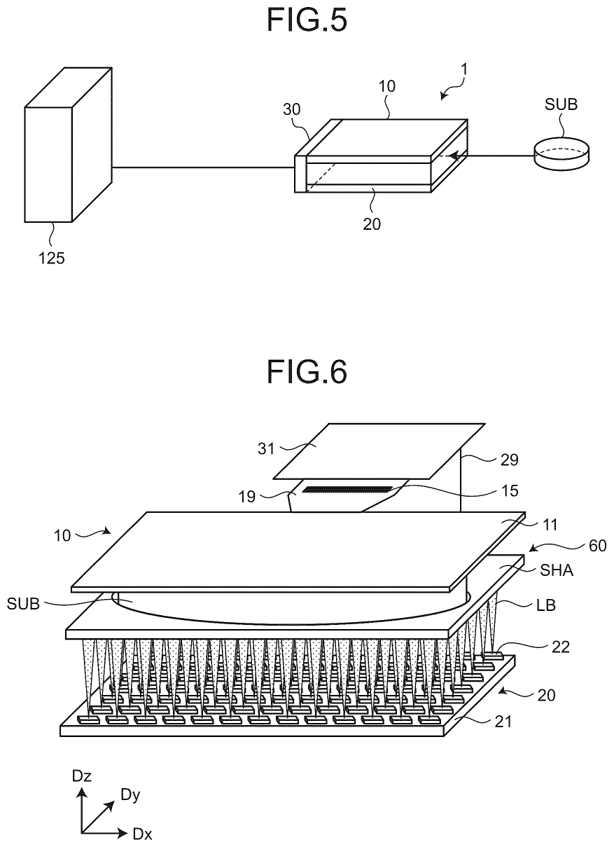

FIG. 5 is a schematic diagram illustrating a relation between one of the detection devices 1 and an external configuration. As illustrated in FIG. 5, the detection device 1 is coupled to the coupling circuit 125 by coupling the control circuit 30 to the coupling circuit 125. As illustrated in FIG. 5, the sensor panel 10 faces the light source panel 20. A gap where the object to be detected SUB can be placed is provided between the sensor panel 10 and the light source panel 20.

The object to be detected SUB is made of a light-transmitting material and has a culture medium (agar) formed on the upper side thereof. The culture medium is a culture medium in which a colony can be cultured. Hereafter, the term simply called “colony” refers to a colony formed by biological tissues or microorganisms cultured in the culture medium formed on the object to be detected SUB. More specifically, the object to be detected SUB is, for example, a glass Petri dish, but is not limited thereto, and may have another configuration that functions in the same way. The culture medium formed on the object to be detected SUB does not have a totally light-blocking property and has such a degree of light-transmitting property that the degree of light transmission varies depending on the presence or absence of the colony and the thickness of the colony.

FIG. 6 is a schematic view illustrating a positional relation between the main configuration of the detection device 1 and the object to be detected SUB. When placing the object to be detected SUB between the sensor panel 10 and the light source panel 20, the object to be detected SUB is placed on a placement member 60, as illustrated in FIG. 6. The placement member 60 serves as a member on which the object to be detected SUB can be placed such that the object to be detected SUB is interposed between the detection area SA and the light source panel 20.

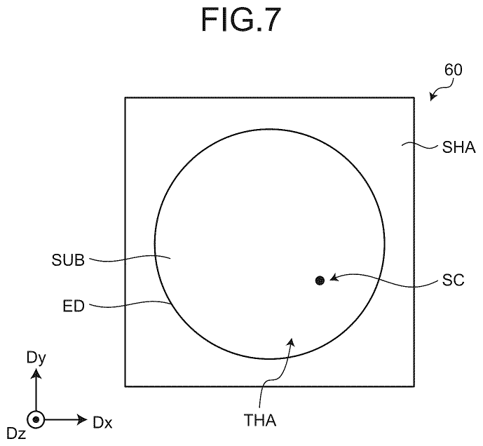

FIG. 7 is a schematic view illustrating an object irradiated by light from the sensor panel 10 in plan view. The planar viewpoint is a viewpoint from which a plane along the first direction Dx and the second direction Dy is directly viewed. The placement member 60 has a configuration including a light-transmitting portion THA and a light-blocking portion SHA. The light-transmitting portion THA, on which the object to be detected SUB is to be placed, is made of a light-transmitting member. The light-blocking portion SHA, which is located around the light-transmitting portion THA as viewed from a planar viewpoint, is made of a light-blocking member. To give a specific example, the light-transmitting portion THA is made of glass or a colorless resin, and the light-blocking portion SHA is made of a black resin. FIG. 7 illustrates the outer edge of the light-transmitting portion THA as an edge ED. That is, with the edge ED as a border, the inside is the light-transmitting portion THA and the outside is the light-blocking portion SHA. The light-transmitting portion THA serves at least partially as a light-transmitting area. The light-blocking portion SHA serves at least partially as a light-blocking area.

For example, when a colony having a shape and size of a colony SC illustrated in FIG. 7 is generated in the culture medium of the object to be detected SUB, the light-transmitting property of the colony SC in the area where the culture medium of the object to be detected SUB is formed is lower than that of an area other than the colony SC.

As illustrated in FIG. 6, the placement member 60 is provided so as to be a member on which the object to be detected SUB can be placed between the sensor panel 10 and the light source panel 20. In the light source panel 20, the light sources 22 are provided on the placement member 60 side of the substrate 21. The light sources 22 are provided so as to emit the light toward the placement member 60. The substrate 11 of the sensor panel 10 is provided such that the detection area SA (refer to FIGS. 1 and 2) faces the placement member 60. The optical sensor WA (refer to FIGS. 2 and 3) in the detection area SA produces an output corresponding to the light that has been emitted from the light source 22 and reached the optical sensor WA through the light-transmitting portion THA, the object to be detected SUB, and the culture medium formed on the object to be detected SUB. Thus, in the embodiment, each of the optical sensors WA produces an output based on the presence or absence and the thickness of the colony generated at a point overlapping the optical sensor WA as viewed from a planar viewpoint.

The light from the light sources 22 at locations facing the optical sensors WA that overlap the light-blocking portion SHA of the placement member 60 as viewed from a planar viewpoint is blocked by the light-blocking portion SHA. Therefore, the optical sensor WA produces an output (lowest output) in a state where virtually no light is detected. In other words, the detection area SA, which is provided with the optical sensors WA two-dimensionally arranged along the first direction Dx and the second direction Dy, has a portion overlapping the light-transmitting portion THA and another portion overlapping the light-blocking portion SHA as viewed from a planar viewpoint. Thus, as viewed from a planar viewpoint, the detection area SA covers both the light-transmitting area (light-transmitting portion THA) and the light-blocking area (light-blocking portion SHA).

The output of each of the optical sensors WA is transmitted to the control circuit 30 via the wiring part 19. FIG. 6 schematically illustrates that the substrate 31 on which the control circuit 30 (refer to FIG. 1) is mounted is coupled to the wiring parts 19 and 29. The wiring part 29 couples the control circuit 30 to the light source panel 20. The wiring parts 19 and 29 are, for example, FPCs, but are not limited thereto, and may have other configurations that function in the same way. The arrangement of the detection circuit 15 schematically illustrated in FIG. 6 is merely exemplary and does not limit the relation between the wiring part 19 and the detection circuit 15. The control circuit 30 illustrated in FIG. 5 is, for example, a schematic representation of the control circuit 30 formed on the substrate 31 illustrated in FIG. 6, and does not indicate the size or shape of the control circuit 30 relative to the sensor panel 10 and the light source panel 20.

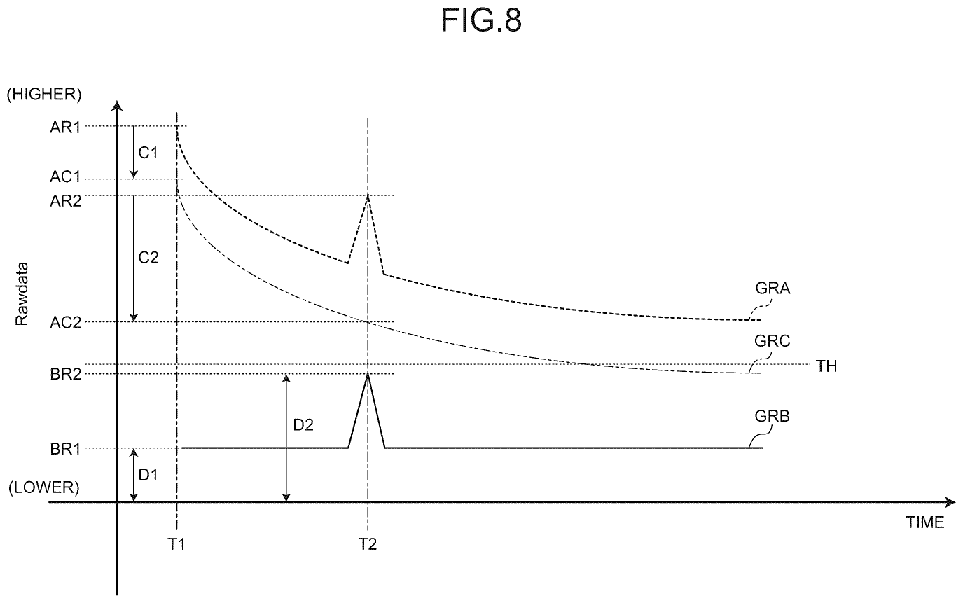

The configuration serving as a prerequisite for the detection of light by the optical sensors WA provided in the detection area SA has been described above with reference to FIGS. 1 to 7. Hereinafter, the term expressed as “output of the detection area SA” refers to the output corresponding to the detection of light by the optical sensors WA provided in the detection area SA. The following describes the output of the detection area SA with reference to FIG. 8.

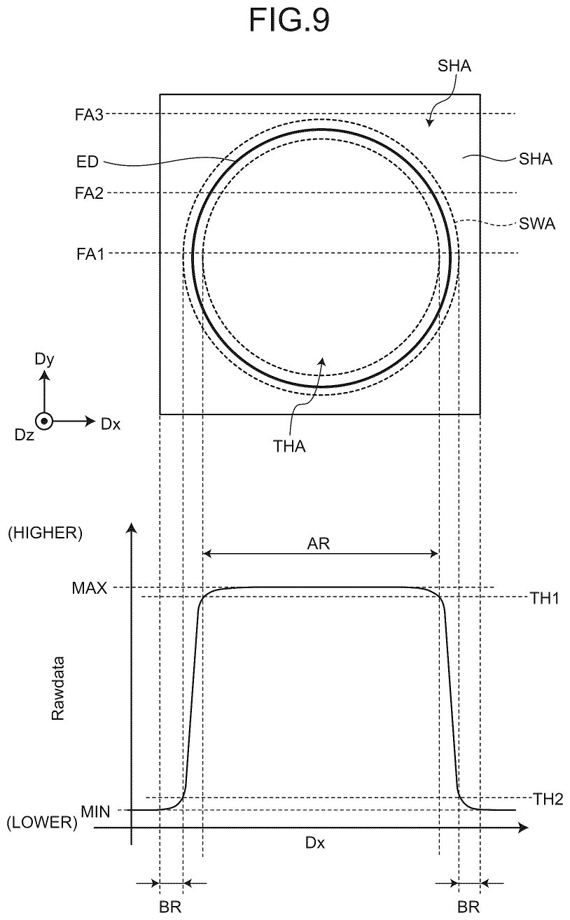

FIG. 8 is a schematic graph illustrating an exemplary relation of the output of the detection area SA with a lapse of time associated with a growth of the colony in the culture medium formed on the object to be detected SUB. “Rawdata” indicated by the vertical axes of the graphs illustrated in FIG. 8 and FIG. 9 to be explained later indicates the output of the detection area SA. The horizontal axes of the graphs illustrated in FIG. 8 indicate time.

As described above, among the optical sensors WA, the optical sensor WA overlapping the light-blocking portion SHA of the placement member 60 as viewed from a planar viewpoint produces an output (lowest output) in the state where virtually no light is detected. FIG. 8 illustrates the lowest output as a graph of a light-blocking area output GRB. In the embodiment, the optical sensor WA indicates even the lowest output as the output at a significantly higher level than when the power is off (no output). FIG. 8 indicates the height of the level of the lowest output as a level D1, relative to no output.

In contrast, the optical sensor WA overlapping the light-transmitting portion THA (refer to FIG. 7) and the object to be detected SUB as viewed from a planar viewpoint produces an output significantly higher than the lowest output. FIG. 8 illustrates the output by the optical sensor WA overlapping the light-transmitting portion THA (refer to FIG. 7) and the object to be detected SUB as viewed from a planar viewpoint as a graph of a light-transmitting area output GRA.

In the culture medium formed on the object to be detected SUB, the colony may be generated over time. The generated colony exhibits changes, such as an increase in area over time, in accordance with the degree of progress of the culture. The graph in FIG. 8 is a graph in the case where such a colony is generated and progressively cultured while expanding over time. Therefore, the output illustrated by the graph of the light-transmitting area output GRA decreases over time, with the exception at time T2. This is because the effect of the colony to block the light traveling from the sensor panel 10 toward the light source panel 20 become more remarkable as the colony is generated and expands.

Unless unintended noise affects the sensor panel 10, the output of the optical sensor WA indicated by the graph of the light-blocking area output GRB remains constant over time, and the output of the optical sensor WA indicated by the graph of the light-transmitting area output GRA decreases with the degree of progress of the culture of the colony generated over time. However, the probability that the unintended noise affects the output of the optical sensor WA is difficult to be reduced to zero. FIG. 8 illustrates the case where the unintended noise has occurred at time T2. At time T2, the outputs of all the optical sensors WA have uniformly increased due to the unintended noise. Therefore, the light-blocking area output GRB at time T2 is at a level D2 that is significantly higher than the level D1, while the light-blocking area output GRB at times other than time T2 is at the level D1. The light-transmitting area output GRA at time T2 is also significantly higher than the output at time points before and after time T2.

If the light-transmitting area output GRA is regarded as the output indicating the degree of progress of the culture of the colony, the output may not accurately indicate the degree of progress of the culture of the colony due to the effect of the unintended noise, as indicated at time T2. Therefore, the embodiment employs a mechanism to reduce the effect of the unintended noise. Specifically, in the embodiment, a process is executed to calculate a difference value obtained by subtracting a first value from a second value. The first value is a value indicating the output level of the optical sensor WA among the optical sensors WA that overlaps the light-blocking portion SHA of the placement member 60 as viewed from a planar viewpoint. The second value is a value indicating the output level of the optical sensor WA among the optical sensors WA that overlaps the light-transmitting portion THA (refer to FIG. 7) and the object to be detected SUB as viewed from a planar viewpoint. The calculation of the difference value corresponds to obtaining a difference between the output of the optical sensor WA overlapping the light-transmitting portion THA and the output of the optical sensor WA overlapping the light-blocking portion SHA. FIG. 8 illustrates the output corresponding to the difference value as a graph of a differential output GRC.

For example, the output level of the light-transmitting area output GRA at time T1 in FIG. 8 is a level AR1. The output level of the light-blocking area output GRB at time T1 in FIG. 8 is a level BR1. The level BR1 is the output level that is higher by the level D1 with respect to the level of no output mentioned above. Therefore, a level AC1 obtained by subtracting the level BR1 from the level AR1 is regarded as the output at time T1. In FIG. 8, the magnitude of a reduction amount C1 indicating a change in direction of reducing the output level from the level AR1 to the level AC1 corresponds to the level D1. The output level of the light-transmitting area output GRA at time T2 in FIG. 8 is a level AR2. The output level of the light-blocking area output GRB at time T2 in FIG. 8 is a level BR2. The level BR2 is the output level that is higher by the level D2 with respect to the level of no output mentioned above. The level D2 has a significantly larger difference from no output than the level D1. A level AC2 obtained by subtracting the level BR2 from the level AR2 is regarded as the output at time T2. In FIG. 8, the magnitude of a reduction amount C2 indicating a change in direction of reducing the output level from the level AR2 to the level AC2 corresponds to the level D2.

In FIG. 8, the light-transmitting area output GRA at time T2 indicates the output level significantly higher than before and after time T2 due to the effect of the unintended noise, which does not accurately reflect the degree of progress of the culture of the colony. In contrast, the differential output GRC including the levels AC1 and AC2 described above indicates a change in which the output level gradually decreases over time. Such a differential output GRC more accurately reflects the degree of progress of the culture of the colony over time. As described above, the process of calculating the difference value can further improve the accuracy of the correspondence between the detection result of the light by the optical sensor WA and the degree of progress of the culture of the colony. The control circuit 30 that calculates the difference value serves as a control circuit that performs processing based on the output of each of the optical sensors WA.

In the embodiment, a determination process is performed to determine, based on the difference value, whether “a colony generated in the culture medium formed on the object to be detected SUB is detected”. To give a specific example, a determination is made as to whether a colony is detected based on the result of comparison of the difference value with a threshold. In the example illustrated in FIG. 8, a threshold TH is illustrated as the output level corresponding to the threshold. If the differential output GRC becomes equal to or lower than the threshold TH, the colony is determined as having been detected. If the differential output GRC exceeds the threshold TH, the colony is determined as not yet having been detected.

The above has described the output of the detection area SA, and the calculation process of the difference value corresponding to the output of the detection area SA, and the determination process. When calculating the difference value, it is necessary to identify the optical sensors WA overlapping the light-transmitting portion THA (refer to FIG. 7) and the object to be detected SUB as viewed from a planar viewpoint and the optical sensors WA overlapping the light-blocking portion SHA of the placement member 60 as viewed from a planar viewpoint.

The area where the light-transmitting portion THA (refer to FIG. 7) and the object to be detected SUB are present as viewed from a planar viewpoint can be regarded as the light-transmitting area where the degree of light-transmitting property changes according to the degree of progress of the culture of the colony. The area where the light-blocking portion SHA of the placement member 60 is present as viewed from a planar viewpoint can be regarded as the light-blocking area where light is blocked. Therefore, the optical sensors WA overlapping the light-transmitting portion THA (refer to FIG. 7) and the object to be detected SUB as viewed from a planar viewpoint can, in principle, be regarded as the optical sensors WA provided in the light-transmitting area. The optical sensors WA overlapping the light-blocking portion SHA of the placement member 60 as viewed from a planar viewpoint can, in principle, be regarded as the optical sensors WA provided in the light-blocking area. However, this principle does not apply to the optical sensors WA that overlap a boundary area SWA to be described later.

In the embodiment, an identification process is performed to identify the optical sensors WA provided in the light-transmitting area and the optical sensors WA provided in the light-blocking area. The object to be detected SUB is not placed on the placement member 60 when the identification process is performed. That is, the object to be detected SUB is not interposed between the sensor panel 10 and the light source panel 20 at the time of the identification process. Except for that fact, the detection of light by the emission of the light and the sensor panel 10 performed for the identification process is performed based on the positional relation among the sensor panel 10, the light source panel 20, and the placement member 60 described with reference to FIGS. 5 and 6.

FIG. 9 is a schematic view illustrating a mechanism for distinguishing the light-transmitting area from the light-blocking area. In FIG. 9, a graph of Rawdata, which illustrates the high-low level of the output of the detection area SA, indicates a change in degree of light transmission along the first direction Dx on a first reference line FA1 of the placement member 60.

As described above, the light-transmitting portion THA has a light-transmitting property and the light-blocking portion SHA has a light-blocking property. Therefore, in a high output area AR overlapping the light-transmitting portion THA on the first reference line FA1, the output of the detection area SA is equal to or almost equal to a maximum output MAX. In a low output area BR overlapping the light-blocking portion SHA on the first reference line FA1, the output of the detection area SA is equal to or almost equal to a minimum output MIN. The maximum output MAX is the output of the optical sensor WA that exhibits the highest output.

The minimum output MIN is the output of the optical sensor WA that exhibits the lowest output. In the embodiment, a set of the optical sensors WA that serve as a basis for obtaining the maximum output MAX and the minimum output MIN corresponds to the optical sensors WA provided in the entire detection area SA, but in modifications to be described later, the maximum output MAX and the minimum output MIN are obtained for each partial area or for each sensor row.

However, in the vicinity of the edge ED, although the output of the detection area SA is higher on the light-transmitting portion THA side and lower on the light-blocking portion SHA side, a difference in level of the output of the detection area SA occurs depending on the position in the first direction Dx. In the vicinity of the edge ED, the output of the detection area SA tends to be not high enough to be said to be equal to the maximum output MAX even on the light-transmitting portion THA side, and the output of the detection area SA tends to be not low enough to be said to be equal to the minimum output MIN even on the light-blocking portion SHA side.

In the embodiment, the optical sensors WA considered to be provided in the light-transmitting area are identified based on a first threshold TH1 with respect to the maximum output MAX and the minimum output MIN. Specifically, the first threshold TH1 corresponds to the output at a first ratio (for example, 95%) when the maximum output MAX is assumed to a 100% output and the minimum output MIN is assumed to be a 0% output. In the embodiment, among the optical sensors WA arranged in the detection area SA, the optical sensor WA producing an output equal to or higher than the first threshold TH1 is identified as the optical sensor WA provided in the light-transmitting area. Therefore, in the example illustrated in FIG. 9, the optical sensor WA in the high output area AR is identified as the optical sensor WA provided in the light-transmitting area. Thus, the output of the optical sensor WA in the high output area AR illustrated in FIG. 9 is reflected to the light-transmitting area output GRA described with reference to FIG. 8.

In the embodiment, the optical sensors WA considered to be provided in the light-blocking area are identified based on a second threshold TH2 with respect to the maximum output MAX and the minimum output MIN. The second threshold TH2 corresponds to an output lower than the first threshold TH1. Specifically, the second threshold TH2 corresponds to an output at a second ratio (for example, 5%) when the maximum output MAX is assumed to be a 100% output and the minimum output MIN is assumed to be a 0% output. In the embodiment, among the optical sensors WA arranged in the detection area SA, the optical sensor WA producing an output equal to or lower than the second threshold TH2 is identified as the optical sensor WA provided in the light-blocking area. Therefore, in the example illustrated in FIG. 9, the optical sensor WA in the low output area BR is identified as the optical sensor WA provided in the light-blocking area. Thus, the output of the optical sensor WA in the low output area BR illustrated in FIG. 9 is reflected to the light-blocking area output GRB described with reference to FIG. 8.

In the embodiment, the optical sensor WA producing an output lower than the first threshold TH1 and exceeding the second threshold TH2 is regarded as the optical sensor WA in the boundary area SWA. The optical sensor WA in the boundary area SWA is the optical sensor WA that overlaps the edge ED as viewed from a planar viewpoint or is located near the edge ED, and is the optical sensor WA that is difficult to be clearly determined, based on the output, whether being provided in the light-transmitting area or provided in the light-blocking area. The output of the optical sensor WA in the boundary area SWA is not used to calculate the difference value. That is, the output of the optical sensor WA in the boundary area SWA is reflected to neither the light-transmitting area output GRA nor the light-blocking area output GRB described with reference to FIG. 8. Therefore, the output of the optical sensor WA that overlaps the boundary area SWA between the light-transmitting area and the light-blocking area is not used to obtain the difference.

The position in the first direction Dx of the optical sensor WA producing the output equal to or higher than the first threshold TH1, as in the high output area AR, depends on the position in the second direction Dy. Also, the position in the first direction Dx of the optical sensor WA producing the output equal to or lower than the second threshold TH2, as in the low output area BR, depends on the position in the second direction Dy. A more detailed description thereof is given below focusing on the first reference line FA1 and a second reference line FA2 positioned closer to an end in the second direction Dy than the first reference line FA1. For example, the linear area overlapping the second reference line FA2 includes a first partial linear area occupied by the optical sensors WA producing the output equal to or higher than the first threshold TH1 and second partial linear areas occupied by the optical sensors WA producing the output equal to or lower than the second threshold TH2. Both ends of the first partial linear area in the first direction Dx are closer to the center in the first direction Dx than those of the first reference line FA1, and each second partial linear area expands toward the center in the first direction Dx with respect to that of the first reference line FA1. FIG. 9 also illustrates a third reference line FA3 positioned closer to the end in the second direction Dy than the second reference line FA2. The third reference line FA3 extends along the first direction Dx and is positioned entirely in the light-blocking portion SHA. The output of any one of the optical sensors WA overlapping the third reference line FA3 is equal to or lower than the second threshold TH2.

The process to identify the optical sensors WA provided in the light-transmitting area and the optical sensors WA provided in the light-blocking area is performed before the object to be detected SUB is placed on the placement member 60, that is, before the start of various processes related to the detection of the colony based on the difference value. In the embodiment, the calculation process of the difference value, the determination process, and the identification process described above are performed by the control circuit 30, but may be performed by other configurations included in the detection device 1, or by an external information processing device coupled to the detection device 1. In that case, the information processing device is regarded as a part of an entire detection device including the configuration of the detection device 1 illustrated in FIG. 1.

In the embodiment, the outputs of the optical sensors WA provided in the detection area SA are classified into outputs of the optical sensors WA provided in the light-transmitting area and outputs of the optical sensors WA provided in the light-blocking area. The outputs of the optical sensors WA provided in the light-transmitting area and the outputs of the optical sensors WA provided in the light-blocking area are averaged separately.

Specifically, the outputs of the optical sensors WA provided in the light-transmitting area are added together, and the sum of the outputs is divided by the number of the outputs of the optical sensors WA provided in the light-transmitting area, thereby obtaining the average value of the outputs of the optical sensors WA provided in the light-transmitting area. In the embodiment, the term “average luminance value of the light-transmitting area” refers to the average value of the outputs of the optical sensors WA provided in the light-transmitting area out of the entire detection area SA. The light-transmitting area output GRA described with reference to FIG. 8 reflects the average luminance value of the light-transmitting area.

The outputs of the optical sensors WA provided in the light-blocking area are added together, and the sum of the outputs is divided by the number of the outputs of the optical sensors WA provided in the light-blocking area, thereby obtaining the average value of the outputs of the optical sensors WA provided in the light-blocking area. In the embodiment, the term “average luminance value of the light-blocking area” refers to the average value of the outputs of the optical sensors WA provided in the light-blocking area out of the entire detection area SA. The light-blocking area output GRB described with reference to FIG. 8 reflects the average luminance value of the light-blocking area.

In the embodiment, an adjustment process is performed based on the brightness of the light from the light sources 22. Specifically, assuming the maximum output MAX to be a 100% output and the minimum output MIN to be a 0% output as described with reference to FIG. 9, the process is performed to handle the output levels of the light-transmitting area output GRA and the light-blocking area output GRB as output levels from 0% to 100%. Thus, the adjustment process is a process to make the output level of the light-transmitting area output GRA correspond to the brightness of the light from the light sources 22. The output level of the light-blocking area output GRB is 0% or very close to 0%.

As described with reference to FIG. 8, the determination process to determine that the “colony generated in the culture medium formed on the object to be detected SUB is detected” is performed based on the difference value and uses a threshold (for example, the threshold TH). The threshold may be a ratio with respect to the maximum output MAX (100%) and the minimum output MIN (0%) on which the adjustment process is based, or may be a predetermined absolute output level. Regardless of which of the methods is used, in an operational standard based on standard setting in advance, experiments performed in advance, and/or the like, the threshold only needs to be set so that the determination result based on the relation between the difference value and the threshold satisfies the operational standard when the culture medium formed on the object to be detected SUB is brought into a pre-assumed “state in which the colony should be regarded to be detected”.

FIG. 10 is a flowchart of processing performed in the detection device 1. When the power of the detection device 1 is turned on (Step S1), the light-transmitting area and the light-blocking area are identified (Step S2). In the process at Step S2, the above-described process to identify the optical sensors WA provided in the light-transmitting area and the optical sensors WA provided in the light-blocking area may be performed, or a mechanism may be employed in which data indicating the result of such an identification process performed before the process at Step S1 is stored in a storage device or a storage circuit provided in the control circuit 30, and the data is read out in the process at Step S2.

After Step S2, the object to be detected SUB is placed (Step S3). In the process at Step S3, the object to be detected SUB is placed on the light-transmitting portion THA of the placement member 60, and the placement member 60 and the object to be detected SUB are placed in the detection device 1 so as to be interposed between the sensor panel 10 and the light source panel 20. The process at Step S3 is performed by human hands, for example, but may be performed mechanically. Before the process at Step S3, the object to be detected SUB is not interposed between the sensor panel 10 and the light source panel 20.

After the process at Step S3, the scan process is performed (Step S4). The scan process is a process to emit the light from the light sources 22, direct the light from the light sources 22 toward the sensor panel 10, and obtain the outputs from the optical sensors WA provided in the detection area SA of the sensor panel 10. By performing the process at Step S4 after the process at Step S3, the scan process is performed with the placement member 60 and the object to be detected SUB interposed between the sensor panel 10 and the light source panel 20. That is, in the outputs obtained in the process at Step S4, the degree of progress of the culture of the colony in the light-transmitting area is reflected to the degree of light transmission.

After the process at Step S4, the adjustment process is applied to the outputs obtained at Step S4 (Step S5). The process at Step S5 is the adjustment process based on the brightness of the light from the light sources 22, as described above.

After the process at Step S5, the difference value between the average luminance value of the light-transmitting area and the average luminance value of the light-blocking area is calculated (Step S6). Specifically, the differential output GRC is obtained as the difference value between the light-transmitting area output GRA and the light-blocking area output GRB, as described with reference to FIG. 8, for example. More specifically, the difference value at a certain time point is calculated in one process at Step S6. For example, at time T1, the calculated difference value is at the level AC1. At time T2, the calculated difference value is at the level AC2.

After the process at Step S6, a determination is made as to whether the difference value indicates the detection of a colony (Step S7). Specifically, the determination process described above with reference to FIG. 8 is performed to determine that the “colony generated in the culture medium formed on the object to be detected SUB is detected” based on the difference value and the threshold (for example, the threshold TH).

If the process at Step S7 determines that the difference value does not indicate the detection of a colony (No at Step S7), the process at Step S4 is performed again. A transition to Step S4 is made after the process at Step S7, whereby the process at Step S4 is performed a plurality of times. The process at Step S4 may be performed at predetermined time intervals. The predetermined time is, for example, 5 minutes, but is not limited thereto, and any time can be employed as the predetermined time.

If the process at Step S7 determines that the difference value indicates the detection of a colony (Yes at Step S7), the control circuit 30 produces an output indicating that the colony has been detected (Step S8). The output by the process at Step S8 is transmitted to the host IC 70 via the coupling circuit 125 and serves as a trigger that causes the host IC 70 to execute a “process to notify an administrator of the object to be detected SUB”. The “process to notify the administrator of the object to be detected SUB” is a process determined in advance, such as a process to send e-mail to a registered mail address of the administrator of the object to be detected SUB, but is not limited to this process. A process may be employed that involves any one or more of all output modes that allow the administrator to be notified that a colony has been detected in the object to be detected SUB.

As described above, according to the embodiment, the detection device 1 includes the sensor panel (sensor panel 10) having the detection area (detection area SA) in which the optical sensors (optical sensors WA) are two-dimensionally arranged, the light sources (light sources 22) that emit light, the member (placement member 60) on which the object to be detected (object to be detected SUB) can be placed such that the object to be detected is interposed between the detection area and the light sources, and the control circuit (control circuit 30) that performs the processing based on the outputs of the optical sensors. The member has the light-transmitting area (light-transmitting portion THA) in which the object to be detected is placed, and the light-blocking area (light-blocking portion SHA) provided around the light-transmitting area. The detection area is located so as to overlap both the light-transmitting area and the light-blocking area. The control circuit obtains the difference between the output of the optical sensor overlapping the light-transmitting area and the output of the optical sensor overlapping the light-blocking area.

With this configuration, even if noise affects the optical sensors (optical sensors WA), the effect of the noise is provided to both the output of the optical sensor overlapping the light-transmitting area (light-transmitting portion THA) and the output of the optical sensor overlapping the light-blocking area (light-blocking portion SHA). Therefore, the difference between the output of the optical sensor overlapping the light-transmitting area and the output of the optical sensor overlapping the light-blocking area is virtually unaffected by the noise. For example, when a denotes the output of the optical sensor overlapping the light-transmitting area and B denotes the output of the optical sensor overlapping the light-blocking area, the difference without the effect of the noise can be expressed as α−β. When γ denotes a change in output due to the effect of the noise, the difference with the effect of the noise can be expressed as (α+γ)−(β+γ)=α−β. Therefore, according to the embodiment, the reduction in accuracy of sensing by the noise can be reduced.

The optical sensor (optical sensor WA) includes the photodiode (PD 82), and therefore, can be relatively faster and more sensitive than other configurations that serve as optical sensors. A two-dimensional detection surface can be more easily formed by arranging the optical sensors.

The outputs of the optical sensors (optical sensors WA) that overlap the boundary area (boundary area SWA) between the light-transmitting area and the light-blocking area are not used to obtain the difference. Thus, the optical sensors overlapping the light-transmitting area can be distinguished from the optical sensors overlapping the light-blocking area. Therefore, the accuracy of the difference can be increased.

### Modifications

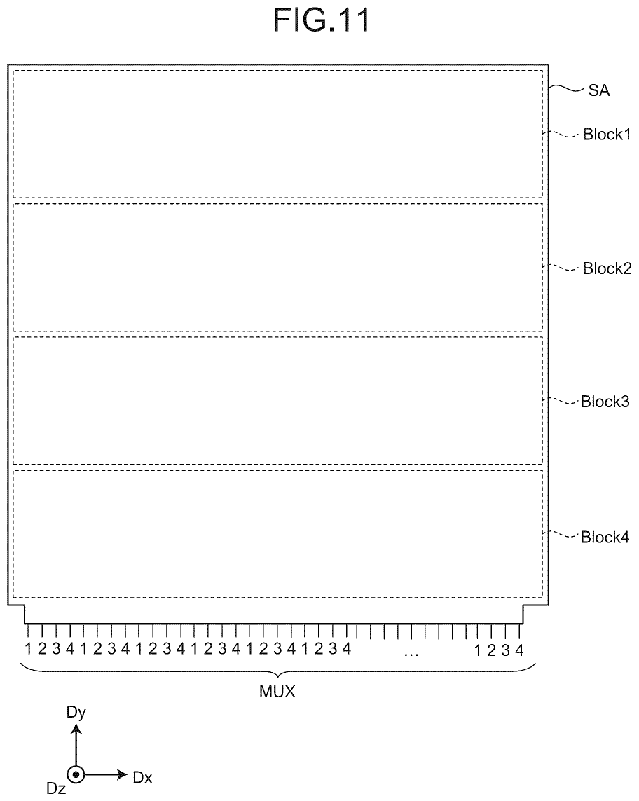

The following describes, with reference to FIGS. 11 to 17, modifications that partially differ from the embodiment described with reference to FIGS. 1 to 10. In the description of the modifications, the same matters as those in the embodiment may be denoted by the same reference numerals and the description thereof may be omitted.

### First Modification

FIG. 11 is a schematic view illustrating an example of the number of blocks in the detection area SA and the number of inputs to each multiplexer. In a first modification of the embodiment, the detection area SA is divided into a plurality of segmented areas (blocks). In the example illustrated in FIG. 11, the detection area SA is divided into a total of four blocks, Block1, Block2, Block3, and Block4. As illustrated as a sequence of blocks Block1, Block2, Block3, and Block4, the blocks segmenting the detection area SA in the first modification are arranged in the second direction Dy. In the first modification, the optical sensors WA provided in the detection area SA are equally or nearly equally divided by the number of the blocks. Each of the blocks includes the same number or nearly the same number of the optical sensors WA.

In the first modification, n is preferably a multiple of the number of blocks. If n is a multiple of the number of blocks, the number of the reset signal transmission lines 5 and the number of the scan lines 6 included in each of the blocks is a number obtained by dividing n by the number of blocks. However, each of the blocks need not include exactly the same number of the optical sensors WA. Some of the blocks may have more of the optical sensors WA than other blocks. The number of the blocks is not limited to four, and only needs to be a natural number equal to or larger than two.

In the first modification, the grouping according to the number of inputs to the multiplexer is further applied. The term “number of inputs to the multiplexer” herein is the number of the switches (such as the switches SW1, SW2, SW3, and SW4) included in the multiplexer 40 described with reference to FIG. 2. FIG. 11 illustrates the grouping according to the number of inputs to the multiplexer by numerics “1”, “2”, “3”, and “4” as multiplexer inputs MUX. The multiplexer input MUX with the numeric “1” indicates the signal line 7 coupled to the switch SW1. The multiplexer input MUX with the numeric “2” indicates the signal line 7 coupled to the switch SW2. The multiplexer input MUX with the numeric “3” indicates the signal line 7 coupled to the switch SW3. The multiplexer input MUX with the numeric “4” indicates the signal line 7 coupled to the switch SW4. In the embodiment and various modifications including the first modification, the number of inputs to each multiplexer is not limited to four, and only needs to be a natural number equal to or larger than two.

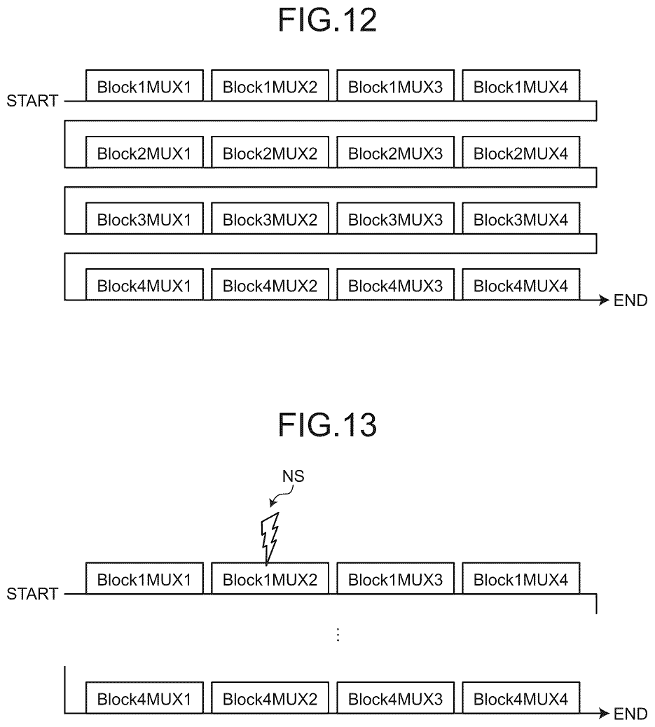

FIG. 12 is a diagram illustrating an exemplary individual detection process based on combinations of the blocks with the inputs to each multiplexer. In the first modification, the outputs of the optical sensors WA in the scan process are grouped based on the combinations of the blocks with the inputs to each multiplexer. FIG. 12 illustrates that the scan process starts from “Block1MUX1”, and the scan process sequentially proceeds in the order of “Block1MUX2”, “Block1MUX3”, “Block1MUX4”, “Block2MUX1”, “Block2MUX2”, “Block2MUX3”, “Block2MUX4”, “Block3MUX1”, “Block3MUX2”, “Block3MUX3”, “Block3MUX4”, “Block4MUX1”, “Block4MUX2”, “Block4MUX3”, and “Block4MUX4”, and ends with the completion of the scan process “Block4MUX4”.

“Block1MUX1” refers to the optical sensors WA included in the block Block1 and refers to the optical sensors WA between which the signal line 7 coupled to the switch SW1 is shared. “Block1MUX2” refers to the optical sensors WA included in the block Block1 and refers to the optical sensors WA between which the signal line 7 coupled to the switch SW2 is shared. “Block2MUX1” refers to the optical sensors WA included in the block Block2 and refers to the optical sensors WA between which the signal line 7 coupled to the switch SW1 is shared. Thus, in the notation of “Block(t)MUX(r)”, (t) is a natural number and takes a value in a range not exceeding the number of blocks. (r) is a natural number and takes a value in a range not exceeding the number of the inputs to the multiplexer. That is, “Block(t)MUX(r)” refers to the optical sensors WA that are included in Block(t) and between which the signal line 7 coupled to the switch SW(r) is shared. In the case of the example illustrated in FIG. 11, (t) and (r) each take any one of values 1, 2, 3, and 4.

In the first modification described with reference to FIG. 11, “Block1MUX1”, “Block1MUX2”, “Block1MUX3”, “Block1MUX4”, “Block2MUX1”, “Block2MUX2”, “Block2MUX3”, “Block2MUX4”, “Block3MUX1”, “Block3MUX2”, “Block3MUX3”, “Block3MUX4”, “Block4MUX1”, “Block4MUX2”, “Block4MUX3”, and “Block4MUX4” cover different partial areas of the detection area SA. For example, “Block1MUX1” can be handled as a partial area of the detection area SA consisting of sensor rows included in the block Block1 and sensor columns coupled to the switch SW1. The total of all outputs of these 16 partial areas is synonymous with the output of the entire detection area SA.

In the process of scanning “Block(t)MUX(r)”, the scan signals are supplied to the scan lines 6 included in the block Block(t) and no scan signals are supplied to the other scan lines 6. In the process of scanning “Block(t)MUX(r)”, the switch SW(r) is turned on (conducting state), and switches other than the switch SW(r) provided in the multiplexer 40 are turned off (non-conducting state). In this way, the output limited to the output from the optical sensor WA indicated by “Block(t)MUX(r)” can be obtained.

According to first modification, performing the scan process described with reference to FIG. 12 can more limit the outputs of the optical sensors WA affected by the unintended noise in the output of the detection area SA. The effect of the noise on the outputs of the optical sensors WA can be reduced more accurately.

FIG. 13 is a diagram illustrating a case where noise NS occurs in the execution of the process illustrated in FIG. 12. In the example illustrated in FIG. 13, the noise NS occurs during the scan process of “Block1MUX2”, and the noise NS does not occur during the other scan processes. In such an example, only the output obtained from the scan process of “Block1MUX2” can be affected by the noise that does not occur in the other scan processes. Therefore, in the first modification, measures against noise only need to be applied only to the output obtained from the scan process of “Block1MUX2” where the noise NS has occurred. Examples of the measures against noise include, for example, the generation of the differential output GRC from the light-transmitting area output GRA by the application of the reduction amount C2 corresponding to the level D2 at time T2 in FIG. 8. This approach makes it easier to reduce the effect of the noise more accurately than in the case where effects of the noise on a portion of the detection area SA are averaged over the entire detection area SA.

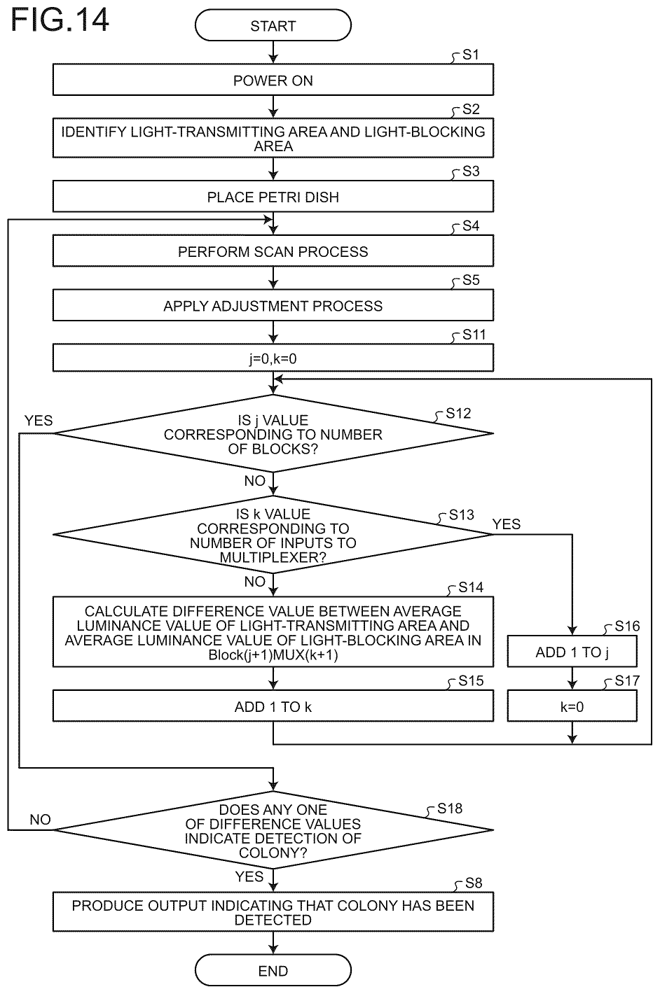

FIG. 14 is a flowchart of processing performed in the detection device 1 according to the first modification. The processing from Step S1 to Step S5 is the same between the embodiment described with reference to FIG. 10 and the first modification to be described with reference to FIG. 14, except in the matter related to the process at Step S4 to be noted below.

In the process at Step S4 in the first modification, the outputs are grouped according to the combination of the blocks and the inputs to each multiplexer, and individual data retention is performed according to the grouped outputs. For example, in the example described with reference to FIGS. 11 and 12, the control circuit 30 retains data individually corresponding to the outputs of the optical sensors WA in each of the total of 16 groups of “Block1MUX1”, “Block1MUX2”, “Block1MUX3”, “Block1MUX4”, “Block2MUX1”, “Block2MUX2”, “Block2MUX3”, “Block2MUX4”, “Block3MUX1”, “Block3MUX2”, “Block3MUX3”, “Block3MUX4”, “Block4MUX1”, “Block4MUX2”, “Block4MUX3”, and “Block4MUX4”. Specifically, the control circuit 30 includes a buffer memory that can store the data. The scan process for generating these pieces of individual data is performed correspondingly to the progression of the scan process in the order described with reference to FIG. 12.

In the first modification, after the process at Step S5, a variable for counting the number of the blocks and a variable for counting the number of the inputs to each multiplexer are set (Step S11). In the example illustrated in FIG. 14, j is set as the variable for counting the number of the blocks. Also, in the example illustrated in FIG. 14, k is set as the variable for counting the number of the inputs to each multiplexer. The variables j and k are set to an initial value of zero.

After the process at Step S11, a determination is made as to whether j is a value corresponding to the number of the blocks (Step S12). For example, in the examples illustrated in FIGS. 11 and 12, the value corresponding to the number of the blocks is four.

In the process at Step S12, if j is determined to be not a value corresponding to the number of the blocks (No at Step S12), a determination is made as to whether k is a value corresponding to the number of the inputs to each multiplexer (Step S13). For example, in the examples illustrated in FIGS. 11 and 12, the number of the inputs to the multiplexer is four.

If the process at Step S13 determines that k is not a value corresponding to the number of the inputs to each multiplexer (No at Step S13), the difference value between the average luminance value of the light-transmitting area and the average luminance value of the light-blocking area in “Block(j+1)MUX(k+1)” is calculated (Step S14). The process at Step S14 is the same as the process at Step S6, except in that the outputs of the optical sensors WA to be processed are limited to “Block(j+1)MUX(k+1)”. For example, if j=0 and k=0, “Block(j+1)MUX(k+1)” is “Block1MUX1”.

After the process at Step S14, one is added to k (Step S15), and the process at Step S12 is performed. Then, if the process at Step S13 determines that k is the value corresponding to the number of the inputs to each multiplexer (Yes at Step S13), one is added to j (Step S16) and the value of k is reset to zero (Step S17). The process at Step S16 and the process at Step S17 may be performed in no particular order. After the processes at Steps S16 and S17, the process at Step S12 is performed.

If the process at Step S12 determines that j is a value corresponding to the number of the blocks (Yes at Step S12), a determination is made as to whether any one of the difference values calculated in a plurality of times of processes at Step S14 indicates the detection of a colony (Step S18). The process at Step S18 is the same as the process at Step S7, except in that a plurality of the difference values are subject to the determination. The number of the difference values corresponds to the number of times by which the process at Step S14 has been performed.

If the process at Step S18 determines that any one of the difference values does not indicate the detection of a colony (No at Step S18), the process at Step S4 is performed again. A transition to Step S4 is made after the process at Step S18, whereby the process at Step S4 is performed a plurality of times. The process at Step S4 may be performed at the predetermined time intervals described above.

If the process at Step S18 determines that one of the difference values indicates the detection of a colony (Yes at Step S18), the process at Step S8 is performed. The first modification is the same as the embodiment, except in the matters noted above.

According to the first modification, the detection area (detection area SA) has the optical sensors (optical sensors WA) arranged in a matrix having a row-column configuration and has a plurality of partial areas. The control circuit (control circuit 30) obtains, for each of the partial areas, the difference between the outputs of the optical sensors overlapping the light-transmitting area (light-transmitting portion THA) and the outputs of the optical sensors overlapping the light-blocking area (light-blocking portion SHA). As a result, the effect of the noise can be reduced more accurately.

### Second Modification

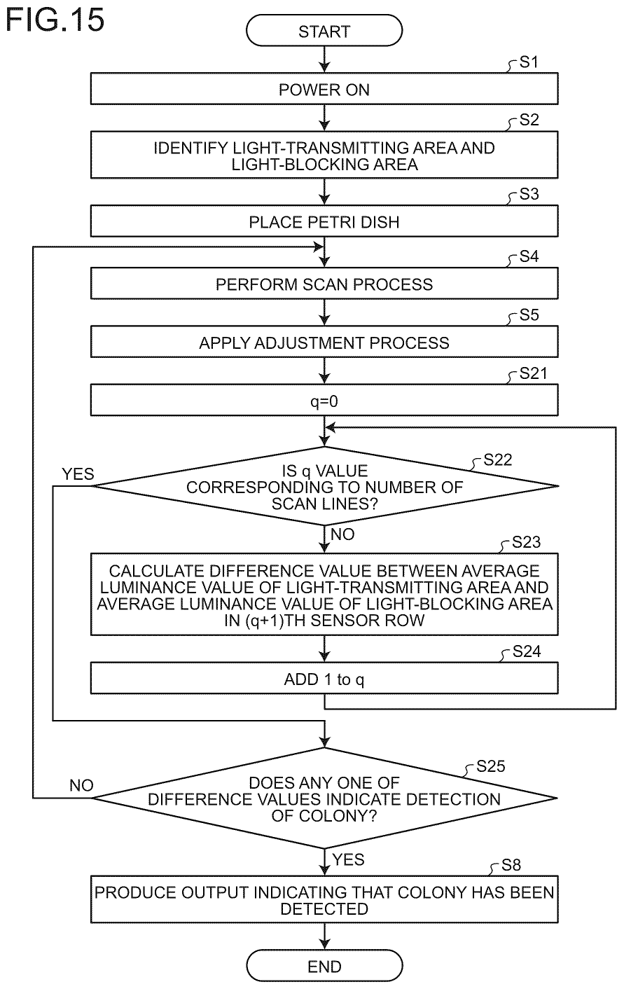

FIG. 15 is a flowchart of processing performed in the detection device 1 according to a second modification of the embodiment. The processing from Step S1 to Step S5 is the same between the embodiment described with reference to FIG. 10 and the second modification to be described with reference to FIG. 15, except in the matter related to the process at Step S4 to be noted below.

However, in the process at Step S4 in the second modification, the outputs are grouped on a scan line 6 basis, and the individual data retention is performed according to the grouped outputs. Therefore, in the second modification, the control circuit 30 retains data individually corresponding to the outputs of the optical sensors WA for each of n sensor rows corresponding to the n scan lines 6. Specifically, the control circuit 30 includes a buffer memory that can store the data.

In the second modification, after the process at Step S5, a variable for counting the number of the scan lines 6 is set (Step S21). In the example illustrated in FIG. 15, q is set as the variable for counting the number of the scan lines 6. q is set to an initial value of zero.

After the process at Step S21, a determination is made as to whether q is a value corresponding to the number of the scan lines 6 (Step S22). For example, in the example illustrated in FIG. 2, the value corresponding to the number of the scan lines 6 is n.

If the process at Step S22 determines that q is not a value corresponding to the number of the scan lines 6 (No at Step S22), the difference value between the average luminance value of the light-transmitting area and the average luminance value of the light-blocking area in the (q+1)th sensor row is calculated (Step S23). The process at Step S23 is the same as the process at Step S6, except in that the outputs of the optical sensors WA to be processed are limited to the (q+1)th sensor row.

After the process at Step S23, one is added to q (Step S24), and the process at Step S22 is performed. Then, if the process at Step S22 determines that q is a value corresponding to the number of the scan lines 6 (Yes at Step S22), a determination is made as to whether any one of the difference values calculated in a plurality of times of processes at Step S23 indicates the detection of a colony (Step S25). The process at Step S25 is the same as the process at Step S7, except in that a plurality of the difference values are subject to the determination. The number of the difference values corresponds to the number of times by which the process at Step S23 has been performed.

If the process at Step S25 determines that any one of the difference values does not indicate the detection of a colony (No at Step S25), the process at Step S4 is performed again. A transition to Step S4 is made after the process at Step S25, whereby the process at Step S4 is performed a plurality of times. The process at Step S4 may be performed at the predetermined time intervals described above.

If the process at Step S25 determines that one of the difference values indicates the detection of a colony (Yes at Step S25), the process at Step S8 is performed.

By calculating the difference value described with reference to FIG. 8 for each of the scan lines 6 as in the second modification, occurrence of streaks due to feedthrough that may occur along with resetting of the optical sensors WA can be more reliably reduced. The streaks due to the feedthrough are caused by the fact that the reset potential of the optical sensor WA may differ depending on the sensor row.

As described with reference to FIG. 3, the potential of the coupling part CP is reset by resetting the output of the PD 82 in response to the signal provided by the reset circuit 13 via the reset signal transmission line 5. Thus, in principle, the potential of the coupling part CP becomes the reset potential VReset. However, the potential after the reset may not be fully unified due to the level of the potential before the reset brought about by various factors such as the degree of detection of light by the PD 82 before the reset. Therefore, if there is a difference in potential after the reset between one and the other of the adjacent sensor rows, the difference may become apparent as a difference in level of the output of the optical sensor WA on a sensor row basis. Such a difference in level of the output of the optical sensor WA between the adjacent sensor rows serves as a difference in brightness when the output of the optical sensor WA is regarded as “detected brightness of light”, as if the sensor rows form light-dark streaks. The streaks due to the feedthrough refers to the occurrence of such a phenomenon.

Therefore, in the second modification, the occurrence of the streaks due to the feedthrough is more reliably reduced by calculating the difference value for each of the scan lines 6, that is, for each sensor row. This is because the calculation of the difference value on a sensor row basis described with reference to FIG. 8 means that the potential of each sensor row after the reset is reflected not only to the high-low level of the light-transmitting area output GRA of each sensor row, but also to the high-low level of the light-blocking area output GRB of each sensor row. In other words, in the second modification, it is possible to handle and reduce, on a sensor row basis, the effect of the reset potential that can differ sensor-row by sensor-row by using both the light-transmitting area output GRA and the light-blocking area output GRB on a sensor row basis, that is, by subtracting the light-blocking area output GRB from the light-transmitting area output GRA. Therefore, the effect of the potential of each sensor row after the reset substantially does not appear in the differential output GRC obtained in the second modification. Thus, according to the second modification, the occurrence of the streaks due to the feedthrough that may occur along with the resetting of the optical sensor WA can be more reliably reduced. The second modification is the same as the embodiment, except in the matters noted above.

According to the second modification, the detection area (detection area SA) has the optical sensors (optical sensors WA) arranged in a matrix having a row-column configuration, and the control circuit (control circuit 30) obtains the difference between the outputs of the optical sensors overlapping the light-transmitting area (light-transmitting portion THA) and the outputs of the optical sensors overlapping the light-blocking area (light-blocking portion SHA), for each row of the optical sensors. As a result, the effect of the streaks due to the feedthrough can be reduced.

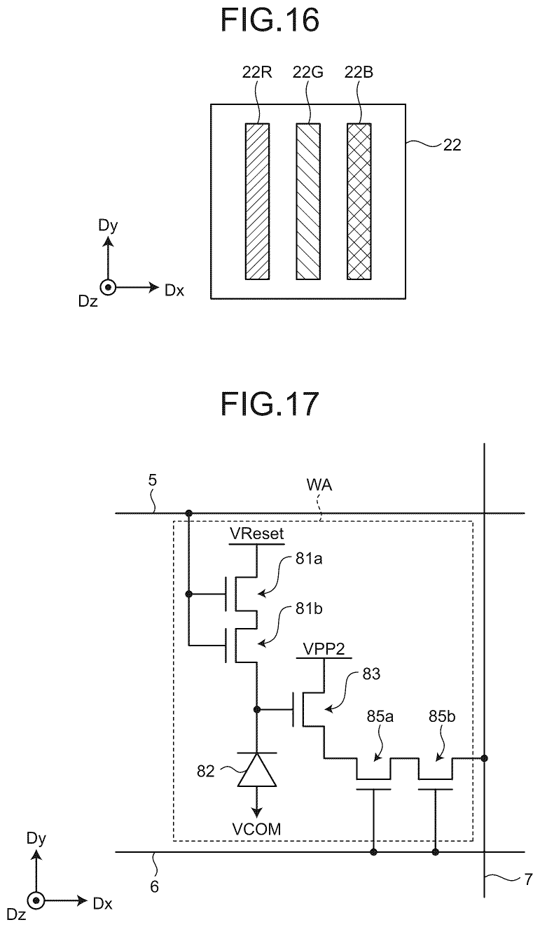

The following exemplifies a configuration common to the embodiment and the various modifications described above, with reference to FIG. 16. FIG. 16 is a schematic view illustrating a configuration example of the light source 22. As illustrated in FIG. 16, the light source 22 includes a first light source 22R, a second light source 22G, and a third light source 22B. The first light source 22R, the second light source 22G, and the third light source 22B are light-emitting elements (such as LEDs) that emit light in different colors. In the embodiment, the first light source 22R emits red (R) light. The second light source 22G emits green (G) light. The third light source 22B emits blue (B) light. When the first light source 22R, the second light source 22G, and the third light source 22B are turned on simultaneously, white light is emitted.

The light source 22 illustrated in FIG. 16 has a configuration in which the longitudinal directions of the first light source 22R, the second light source 22G, and the third light source 22B are along the second direction Dy, and the first light source 22R, the second light source 22G, and the third light source 22B are arranged in this order from one side toward the other side in the first direction Dx. This configuration is, however, an exemplary form of the light source 22, which is not limited to this form. For example, the shape of the first light source 22R, the second light source 22G, and the third light source 22B in the light source 22 as viewed from a planar viewpoint and the positional relation among the first light source 22R, the second light source 22G, and the third light source 22B can be changed as appropriate. A single white light source may be provided instead of the first light source 22R, the second light source 22G, and the third light source 22B.

The switching elements 81 and 85 illustrated in FIG. 3 are each not limited to the configuration with a single switching element. FIG. 17 is a circuit diagram illustrating a circuit configuration of the optical sensor having a partially different configuration from that of FIG. 3. For example, the switching element 81 may have what is called a double-gate configuration with switching elements 81a and 81b, as illustrated in FIG. 17. The switching element 85 may also have what is called a double-gate configuration with switching elements 85a and 85b, as illustrated in FIG. 17.

The object to be detected, such as the object to be detected SUB, is not limited to the Petri dish on which the culture medium is formed, and may have another configuration. The object to be detected may be, for example, a plate for suspension culture.

The arrangement of the optical sensors WA is not limited to the matrix arrangement along the first direction Dx and the second direction Dy. For example, the optical sensors WA arranged in the sensor rows adjacent in the second direction Dy need not both be located on a straight line along the second direction Dy. Specifically, the optical sensors WA may be located in what is called a staggered manner. From the viewpoint of sharing the reset signal transmission line 5 and the scan line 6, the arrangement of the optical sensors WA in the first direction Dx is preferably such that the optical sensors WA are located on a straight line along the first direction Dx, but this arrangement is also not essential, and can be changed as appropriate within a range of not hindering the functions of the optical sensors WA and the detection area SA. The arrangement of the light sources 22 in the light source panel 20 is also not limited to the matrix arrangement, and can be any arrangement.

Other operational advantages accruing from the aspects described in the present embodiment that are obvious from the description herein, or that are conceivable as appropriate by those skilled in the art will naturally be understood as accruing from the present disclosure.

What is claimed is:

1. A detection device comprising:
a sensor panel that has a detection area in which a plurality of optical sensors are two-dimensionally arranged;
a light source configured to emit light;
a member on which an object to be detected is to be placed such that the object to be detected is interposed between the detection area and the light source; and
a control circuit configured to perform processing based on outputs of the optical sensors, wherein
the member has a light-transmitting area in which the object to be detected is to be placed and a light-blocking area provided around the light-transmitting area,
the detection area is located so as to overlap both the light-transmitting area and the light-blocking area, and
the control circuit is configured to obtain a difference between the outputs of the optical sensors overlapping the light-transmitting area and the outputs of the optical sensors overlapping the light-blocking area.

2. The detection device according to claim 1, wherein the optical sensors comprise photodiodes.

3. The detection device according to claim 1, wherein
the detection area comprises:
the optical sensors arranged in a matrix having a having a row-column configuration; and
a plurality of partial areas, and
the control circuit is configured to obtain, for each of the partial areas, the difference between the outputs of the optical sensors overlapping the light-transmitting area and the outputs of the optical sensors overlapping the light-blocking area.

4. The detection device according to claim 1, wherein
the detection area comprises the optical sensors arranged in a matrix having a row-column configuration, and
the control circuit is configured to obtain the difference between the outputs of the optical sensors overlapping the light-transmitting area and the outputs of the optical sensors overlapping the light-blocking area, for each row of the optical sensors.

5. The detection device according to claim 1, wherein the outputs of the optical sensors that overlap a boundary area between the light-transmitting area and the light-blocking area are not used to obtain the difference.
